(19) United States
(12) Patent Application Publication
(10) Pub. No.: US 2012/0089081 A1
(43) Pub. Date: Apr. 12, 2012

(54) Piezoelectric element driver

(76) Inventors: Wahng Chao Uei, Fremont, CA (US); Hieu Tieu, San Jose, CA (US); Yuchen Zhou, San Jose, CA (US)

(21) Appl. No.: 13/317,203

(22) Filed: Oct. 11, 2011

Related U.S. Application Data
(60) Provisional application No. 61/404,922, filed on Oct. 12, 2010.
(60) Provisional application No. 61/456,164, filed on Nov. 2, 2010.

(51) Int. Cl. A61M 35/00

(52) U.S. Cl. 604/22

(57) ABSTRACT

The invention teaches a palm-held massage device which includes a driver that drives one or more massage elements which provides massage vibrations in one or more ultrasonic and subsonic frequencies, a controller that provides commands to the driver, a signal generator that provides frequency references to the driver, a power supply coupled to a contactless charger, and a specimen dispensing system.

### REFERENCE TO RELATED APPLICATIONS

The present application claims priority to the provisional Appl. Ser. No. 61/404,922 filed on Oct. 12, 2010, the entire content of which is hereby incorporated by reference. The present application is also related to the provisional Appl. Ser. No. 61/456,164 filed on Nov. 2, 2010, the entire content of which is hereby incorporated by reference.

### FIELD OF THE INVENTION

The present invention relates generally to ultrasonic technology. More particularly, it relates to a piezoelectric element driver incorporated in a palm-held ultrasonic skin care device which has a built-in integrated skin-treatment specimen dispenser along with an electrical interface to enable easy application of the specimen for hygiene operation and skin beautification purposes.

### BACKGROUND OF THE INVENTION

It has been well realized that ultrasonic technologies can be used in skin treatment and beautification. Traditionally, ultrasonic devices using piezo transducers require a resonant driver which utilizes a transistor with the piezo transducer itself acting as an oscillator while the surrounding circuits amplify the signal and feedback onto itself. While the frequency target of this method is very good, it drains a lot of power since the components in the circuits surrounding the piezo transducer all consume too much power and the electrical power delivered to the piezo transducer itself is very inefficient. FIG. 1A is a circuit diagram illustrating a colpitt's oscillator for driving a piezo transducer according to the prior art. In FIG. 1A, C3 and L1 form a resonating tank circuit, where C1 is the piezo device.

FIG. 1B is a schematic diagram illustrating a higher level system configuration of the piezo transducer in FIG. 1A and how it works. The piezo transducer unit 1 forms a feedback loop circuit with an oscillator sub-circuit and an amplifier. The major problem of such design is that the oscillator circuit components consume too much power during operation and makes the design not energy efficient, especially for miniature handheld devices operating on small size batteries.

What is desired is a driving system wherein each component can be individually optimized or changed without affecting the performance of other components, which is not possible in prior art due to the coupled resonance between the piezo transducers and other components.

What is further desired is that without resonator tank, the power supplied to the driving system is efficiently channeled from the power supply to the massage elements such as piezo transducers in a single direction without being wasted in other components.

### SUMMARY OF THE INVENTION

The present invention teaches a palm-held massage device which includes a driver that drives one or more massage elements, called piezo transducers or ultrasonic transducers, which provides massage vibrations in one or more ultrasonic and subsonic frequencies approximately between 10 KHZ and 100 MHZ, a controller that provides commands to the driver, a signal generator that provides frequency references to the driver, a power supply coupled to a contactless charger, and a specimen dispensing system.

The specimen dispensing system includes one or more cartridges coupled to an array of dispensers embedded in the surface of the massage elements, wherein the array of dispensers are controlled by the controller. The specimen dispensed by the dispensers flows to the surface of the massage elements from one or more outlets.

The signal generator provides reference frequencies. The massage elements coupled to the generator provide vibrations at frequencies approximately between 10 KHZ and 100 MHZ. The vibrations are applied to human skin through a treatment plate which is an exterior member of the device's body. The frequency reference signals are electrically decoupled from the ultrasonic transducers.

The signal generator is a semiconductor circuit comprising a first inverter, a second inverter and a first capacitor that are electrically coupled in series, and an adjustable resistor electrically coupled to a first node between the first and the second inverters and to a second node between the second inverter and the first capacitor.

In particular, the signal generator is a semiconductor circuit that includes: (1) a non-inverting operational amplifier (NOA) with its input terminal coupled to a first feedback resistor via a third node, with its output terminal coupled to a fourth node and with its feedback terminal coupled to a second capacitor for AC coupling via a fifth node; (2) a second feedback resistor coupled between the fourth node and the third node, said third node being between the amplifier's input terminal and the first resistor; and (3) a third feedback resistor coupled between the fifth node and the amplifier's feedback terminal.

The driver is a semiconductor circuit that includes: (1) an input node; (2) a sixth node; (3) an output node; (4) an inverter coupled between said input node and the sixth node, the inverter comprising an n-type MOSFET and a p-type MOSFET with their gate terminals coupled to the input node and their source terminals coupled to the sixth node; (5) an amplifier coupled between the sixth node and the output node; and (6) an inductor coupled between the output node and a power supply.

The amplifier can be an n-type MOSFET or a bipolar transistor.

### BRIEF DESCRIPTION OF THE DRAWINGS

FIG. 1A is a circuit diagram illustrating a driver used in an ultrasonic device according to prior art;

FIG. 1B is a schematic block diagram illustrating a typical driver having a feedback loop according to prior art;

FIG. 2 is a schematic block diagram illustrating a driver according to the present invention;

FIG. 3 is a schematic block diagram illustrating a driving system having a feedback loop according to the preferred embodiment of the present invention;

FIG. 4 is a circuit diagram illustrating a typical signal generator used in the driving system according to the present invention;

FIG. 5 is a diagram illustrating the oscillating signals generated by the generator of FIG. 4;

FIG. 6 is a circuit diagram illustrating a signal generator which includes a feedback network;

FIG. 7 is a diagram illustrating the output of the circuit of FIG. 6;

FIG. 8 is circuit diagram illustrating a main driver of FIG. 3;

FIG. 9 is a circuit diagram illustrating a typical boost converter used as power supply for the driving system of FIG. 3;

FIG. 10 is a circuit diagram illustrating a power supply system used in the driving system of FIG. 3;

FIG. 11 is a circuit diagram illustrating a contactless coupling between a charger and the power supply system;

FIG. 12A is a circuit diagram illustrating a piezo transducer without a feedback to the signal generator; and

FIG. 12B is a circuit diagram illustrating a piezo transducer with a feedback to the signal generator.

### DESCRIPTION OF THE INVENTION

While the present invention may be embodied in many different shapes, forms, designs or configurations, for the purpose of promoting an understanding of the principles of the invention, reference will be made to the embodiments illustrated in the drawings and specific language will be used to describe the same. It will nevertheless be understood that no limitation of the scope of the invention is thereby intended. Any alterations and further implementations of the principle, the essence or the spirit of the invention as described herein are contemplated as would normally occur to one skilled in the art to which the invention relates.

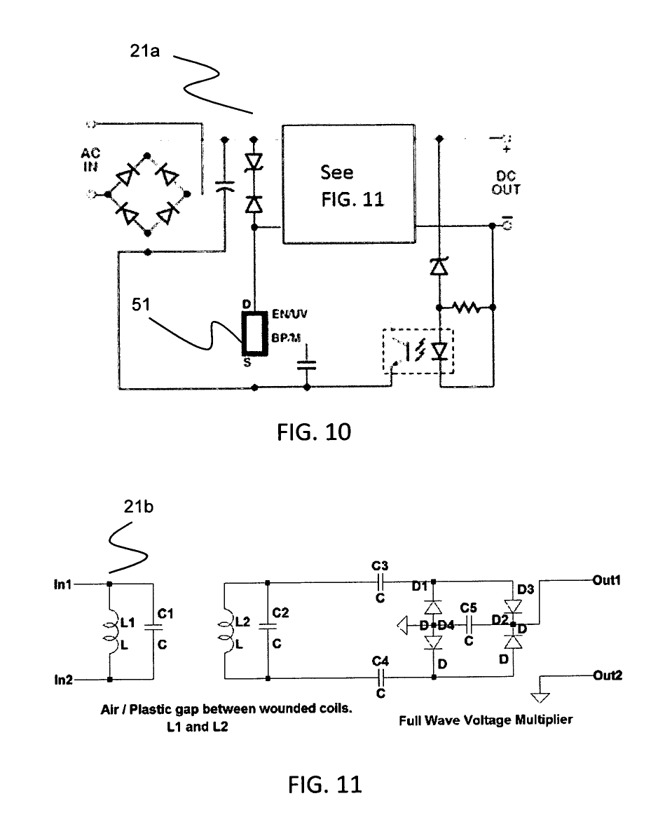

This invention teaches a palm-held massage device which includes a driver that drives one or more massage elements which provides massage vibrations in one or more ultrasonic and subsonic frequencies at frequencies approximately between 10 KHZ and 100 MHZ, a controller that provides commands to the driver, a signal generator that provides frequency references to the driver, a power supply coupled to a contactless charger, and a specimen dispensing system. The frequency reference signals are electrically decoupled from ultrasonic transducers. In this invention, “contactless charger” means that the power supply within the device can be charged without a conductive wire connected to the source of power. In a typical configuration, the electrical energy is conveyed from a first inductor to a second conductor as illustrated in FIG. 10 and FIG. 11.

The specimen dispensing system includes one or more cartridges coupled to an array of dispensers embedded in the surface of the massage elements, wherein the array of dispensers are controlled by the controller. The specimen dispensed by the dispensers flows to the surface of the massage elements from one or more outlets.

The signal generator provides reference frequencies. The massage elements, also called piezo transducers, coupled to the generator provide ultrasonic vibrations. The frequency reference signals are electrically decoupled from piezo transducers. The vibrations are applied to human skin through a treatment plate which is an exterior member of the device's body. Specifically, the treatment plate has a smooth exterior surface for touching human skin. There are one or more piezo transducers are coupled to the interior side of the treatment plate. The ultrasonic vibrations at frequencies approximately 10 KHZ-100 MHZ from the piezo transducers are transmitted to the target skin are through the treatment plate. In an implementation with multiple piezo transducers coupled to an integrated treatment plate, the piezo transducers can be operated alternatively with different frequencies and a time sequence according the settings of a micro-controller. For example, transducer 1 operates at frequency 1, transducer 2 at frequency 2, transducer 3 at frequency 3, . . . then transducer 1 at frequency 1, transducer 2 at frequency 2, so on and so forth.

In a typical implementation, the signal generator is a semiconductor circuit comprising a first inverter, a second inverter and a first capacitor that are electrically coupled in series, and an adjustable resistor electrically coupled to a first node between the first and the second inverters and to a second node between the second inverter and the first capacitor.

In particular, the signal generator is a semiconductor circuit that includes: (1) a non-inverting operational amplifier (NOA) with its input terminal coupled to a first feedback resistor via a third node, with its output terminal coupled to a fourth node and with its feedback terminal coupled to a second capacitor for AC coupling via a fifth node; (2) a second feedback resistor coupled between the fourth node and the third node, said third node being between the amplifier's input terminal and the first resistor; and (3) a third feedback resistor coupled between the fifth node and the amplifier's feedback terminal.

The driver is a semiconductor circuit that includes: (1) an input node; (2) a sixth node; (3) an output node; (4) an inverter coupled between said input node and the sixth node, the inverter comprising an n-type MOSFET and a p-type MOSFET with their gate terminals coupled to the input node and their source terminals coupled to the sixth node; (5) an amplifier coupled between the sixth node and the output node; and (6) an inductor coupled between the output node and a power supply.

The circuit design according to this invention can efficiently channel most of the electrical energy into driving the target piezo transducers, which in turn generate the most amount of mechanical energy possible. It does not use resonance formed by the piezo transducers and other sub-circuits as its primary drive. Rather, a low cost oscillating signal generation component is used to generate the AC voltage/current signal that couples to the piezo transducers with the correct frequency without using the power consuming electronic components as in prior art.

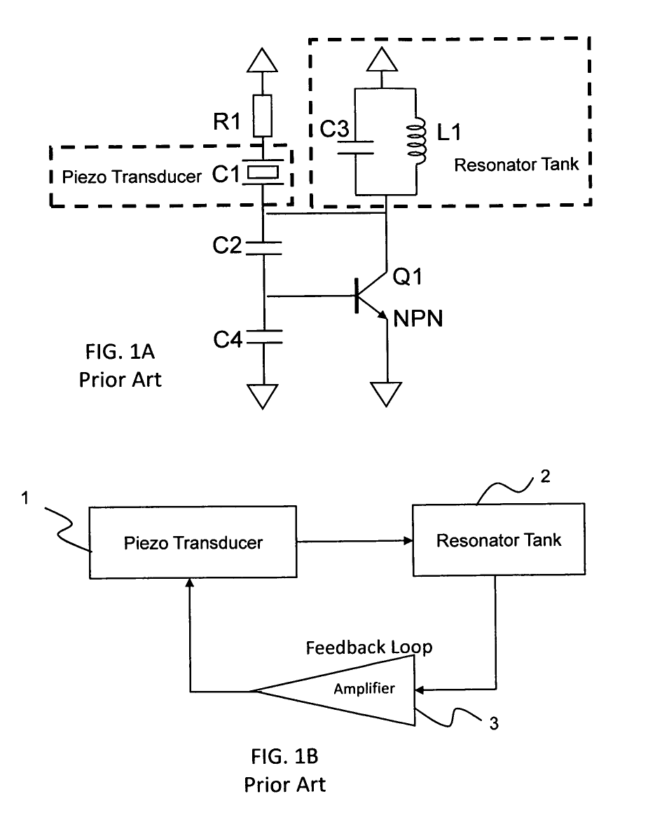

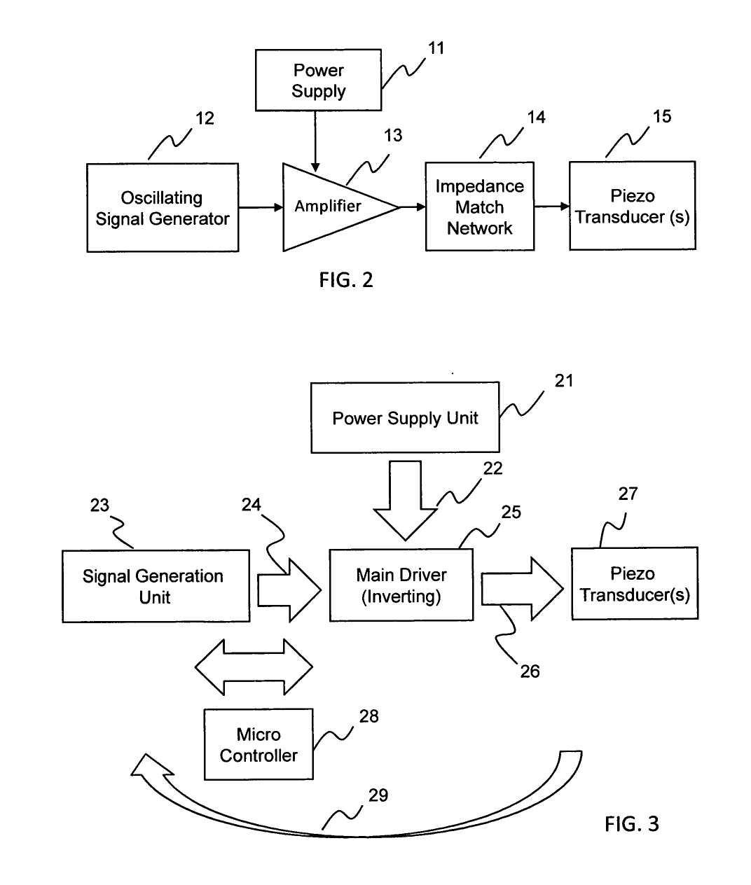

FIG. 2 is a schematic block diagram illustrating a typical driver according to the present invention. The driver includes a power supply 11, an oscillating signal generator 12, a signal amplifier 13, an impedance match network 14 and Piezo Transducers 15. One advantage of this design is that each component can be individually optimized or changed without affecting the performance of other components, which is not possible in prior art design due to the coupled resonance between the piezo transducers and other components. Another key advantage is that without a resonator tank as in prior art as shown in FIG. 1A and FIG. 1B, the power is efficiently channeled from the power supply 11 to the piezo transducer(s) 15 in a single direction without being wasted in other component. Note that the impedance match network 14 is an optional component in this invention.

FIG. 3 is a system level schematic block diagram illustrating a driving system according to the preferred embodiment of the present invention. The system includes a power supply unit 21, a signal generation unit 23, a main driver 25, a group of piezo transducers 27, a micro-controller 28, and an optional feedback loop 29. The power supply unit 21 is electrically coupled to the main driver 25 through a circuit 22. The oscillating signal from the signal generation unit 23 is provided to the main driver 2 through a circuit 24. The electrical driving energy from the main driver 25 is delivered to the piezo transducers 27 through a circuit 26. All of these components are incorporated inside of device body. In a typical implementation, the device body consists of two members coupled together, each of which having a smooth exterior surface. The piezo transducers are coupled to the inner side of the first member which provides vibrations at frequencies approximately between 10 KHZ and 100 MHZ. to the target skin area. The second member is the backside cover of the device body.

### Signal Generation Unit

The driving system illustrated in FIG. 3 requires an input signal 24, which can be a logical TTL signal or an analog signal. The input signal 24 drives over the input threshold of the main driver 25. There are two ways to supply signal to the main driver 25 cost-effectively and efficiently. One of which can be standalone. The other one can be adjusted by a feedback network.

Option 1: For low cost and simple oscillating signal generation, a crystal oscillator component with desired frequency that matches to the oscillating frequency of the piezo transducer 27 can be used.

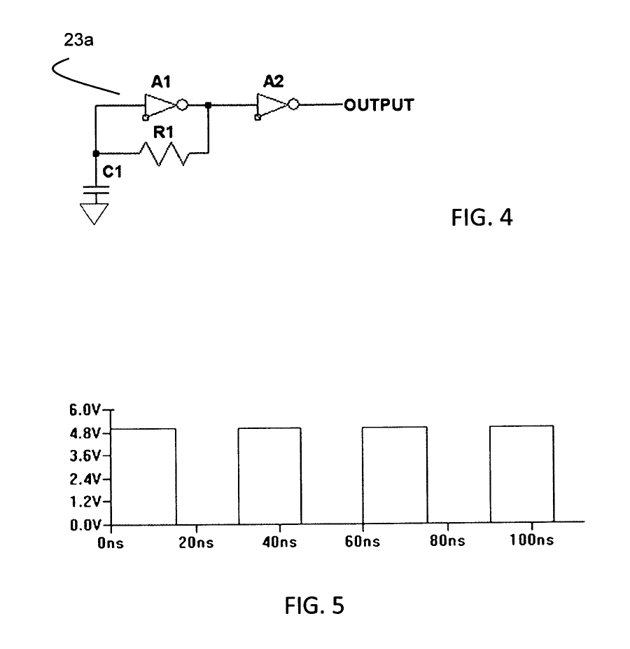

Option 2: As illustrated in FIG. 4, the signal generator 23a can be two inverters A1 and A2 coupled in a feedback loop. The inverters A1 and A2 have to be fast enough for the target frequency. RI is an adjustable resistor to tune the frequency and C1 is in the RC network to slow down the clocks. Output of the generator of FIG. 4 is a square wave as illustrated in FIG. 5.

Option 3: A microcontroller output can be used for an oscillating signal generation.

This type of simple signal generator 23a as shown in FIG. 4 does not need a feedback signal from the piezo 27 in FIG. 3. Instead, it generates its own signal from an inverter feedback represented by R1 in FIG. 4.

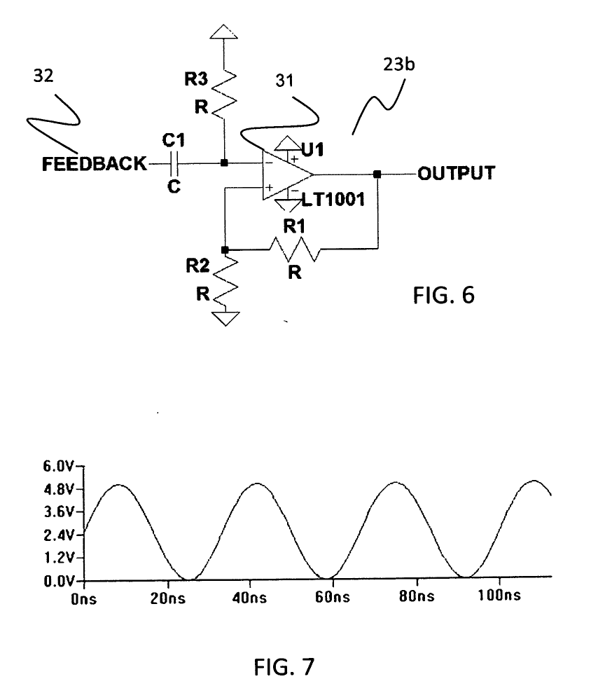

FIG. 6 is a circuit diagram illustrating a signal generator which includes a feedback network. The signal generator 23b includes a low bias input op-amp 31, which is already in a negative feedback configuration (R1 and R2). The amplification of op-amp 31 depends on the feedback signal 32 from the piezo 27 of FIG. 3, which must have enough gain to cross the threshold for the main driver 25. In a typical configuration, a gain of 3 times is sufficient. In FIG. 6, R3 is for bias and C1 is for AC coupling. By using an optional feedback path 29, the frequency can be tuned automatically and accurately. However, different from the feedback loop in prior art as shown in FIG. 1B, the feedback path 32 as illustrated in FIG. 6 consumes very little power since it is not for oscillation generation as in prior art.

### Main Driver

Refer to FIG. 3, the main driver 25 determines how much voltage/current, and at what frequency the piezo transducer 27 can be driven.

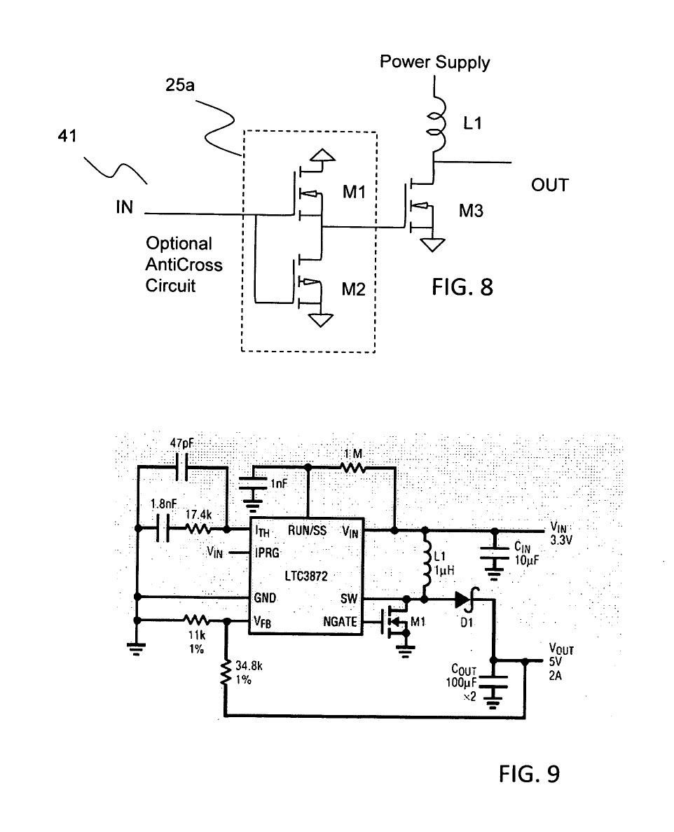

FIG. 8 is circuit diagram illustrating a main driver of FIG. 3. The main driver 25a includes M1 (an N-MOS) and M2 (a P-MOS) coupled together to act as an inverter. The M1 and M2 inverter can be replaced with a MOSFET driver. The signal input 41 translates to a very hard pull to Vcc or Gnd on the gate of M3. These devices can be complicated to include anti cross circuits to further reduce power consumption. M3 can be replaced by a bipolar transistor. In fact, the whole circuit (M1, M2, and M3) can be replaced by a more powerful transistor. However, that would not be cost effective. L1 inductor is a critical component as it stores the energy needed to deliver to the piezo device. The energy storage and current capability of L1 determine the amount of energy being sent to the piezo transducer 27. This is similar to but not the same as a class E amplifier, where the capacitive element is removed and replaced with a piezo instead. It is also similar to a boost converter and the voltage is boosted at the output. Such a driver is capable of driving multiple piezo transducers at the same time, an advantage for increasing the vibration surface.

### Power Supply Unit

A high voltage supply is usually needed for operation of piezo transducers. However, in small-size portable implementation, we have to rely on smart circuitry. So, only a boost of 10V is necessary. Coming from a battery voltage, we can step up using a standard boost converter like one as shown in FIG. 9.

Refer to FIG. 3, the power supply unit 21 may include a charger. Charging the battery that supplies the electrical power can be realized by a contactless charging component. This not only leverages existing infrastructure for power supply designs, which has all the nessasary safety built in and FCC approvals, but also makes it extremely cheap to produce. Using a battery protection IC meant for lithium ion systems (3.3-4.2V), we can protect NiMH or other chemistry of batteries in a stack. Since the voltage of 4×NiMH batteries are 4.8V, a 4.2V protection can be used. This can potentially extend the battery life at the expense of power cutoff at low charge.

FIG. 10 is a circuit diagram illustrating a power supply system 21a used in the driving system of FIG. 3. FIG. 11 is a circuit diagram illustrating a contactless coupling between a charger 21b and the power supply system. Inductor L1 and capacitor C1 have to be tuned to the frequency that the supply switcher 51 is on. They also have to hold the high voltage from the supply line. Similarly, inductor L2 and capacitor C2 also need to be tuned to the frequency. However, the interaction between L1 and L2 affects the inductances of each other, making tuning difficult. L1 and L2 can be made from wounded coils and can be used with an iron core at a close range such as 4 mm, or simply a flat wound coil. Tuning the frequency is critical because L1 and L2 need to match one another to get more efficient. The second portion of the circuit (full wave voltage multiplier) boosts the voltage up and also rectifies it at the same time, providing a DC output. This output can then be used to charge the battery. This method is safe and efficient since no transformers are necessary (they are in coils now), which makes everything isolated and away from dangerous voltages.

### Piezo Transducers

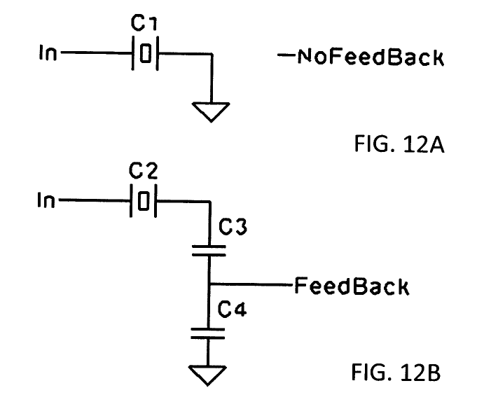

Refer to FIG. 3, multiple resonating piezo transducers with different oscillating frequencies may be used in the system, wherein different frequency piezo transducers are driven by the same driving circuit at different time slots with switching between different frequencies controlled by the micro-controller 28. To generate feedback from the different frequency transducers for use as in the circuit illustrated in FIG. 6, after the main driving power has passed through the piezo transducer C1, a small signal remains on the other side. As illustrated in FIG. 12B, this can be extracted through a couple of series connected low capacitance capacitors C3 and C4 connected to the negative end, then feedback the signal to the signal generator 23 of FIG. 3. Optionally, the other side of the piezo transducer can be simply grounded without a feed back as illustrated in FIG. 12A.

### Micro Controller

Refer to FIG. 3, the micro controller 28 is a firmware having various functions. It monitors the battery voltage and displays warnings. It enters into charge mode, non-charge mode, or a power saving mode. It processes the capacitive touch control and power-up mode and power-down mode accordingly. Furthermore, it controls the “on” and “off” functions of the signal generation unit 23, and thus switching between different oscillating frequency piezo transducers, or driving them at different time according to a pre-set sequence.

Since each main driver is capable of driving multiple piezos, the power is distributed among the number of piezos driven. Thus, in this type of architecture, we can effectively increase the maximum power transfer to each transducer by using multiple drivers, and multiplexing each driver in time. Each driver can then deliver the full available power while maintaining a low average power usage by maintaining a low gap between each burst.

The micro-controller 28 may include an embedded data storage device for storing operational information. It is capable of displaying the information to a user through visual, skin contact or sound effects and is capable of operating the skin treatment plate in a specific manner determined by data stored in the storage device. The operational information can be, but not limited to, operation data, user skin condition data, user personal information, and cartridge identification data.

The micro-controller 28 also controls the dispensing of specimen such as lotion from one or more cartridges incorporated in the device's body.

### Device's Body and Integration

In a typical implementation of this invention, the device has an integrated body in an ellipsoidal shape or other shape with curved and smooth exterior surfaces. The device body includes a first member and a second member mechanically coupled together. The first member's and second member's shapes are preferably symmetrical. The first member is a treatment plate for contacting the target skin area. One or more piezo transducers are coupled to the backside of the treatment plate. The specimen outlet is on the treatment plate. It can be one or more small round holes with a diameter less than 1 mm. The outlet is coupled to a cartridge containing specimen such as lotion to be dispensed to the target skin area. The second member is a backside cover.

The driver, the controller, the signal generator, the power supply and the dispensing system including the cartridge are electrically and communicatively coupled together and all physically enclosed inside of the device's body. “Communicatively” here means that the controller is capable of acquiring data from other components and the other components are capable of receiving signals or commands from the controller.

The specimen can be liquid, gel, serum, cream, paste and powder. The cartridge can be implemented as multiple sub-cartridges containing same or different specimens and the sub-cartridges can be individually selected to dispense specimen therein. The cartridge may have multiple specimen compartments containing same or different specimens and each of the compartments can be individually selected to dispense specimen therein. To replace battery, specimen or cartridge, the user may open the backside cover easily, install the needed, and then cover it.

While one or more embodiments of the present invention have been illustrated above, the skilled artisan will appreciate that modifications and adoptions to those embodiments may be made without departing from the scope and spirit of the present invention.

1. A palm-held massage device for skin treatment by concurrently applying ultrasonic vibrations and dispensing a specimen such as lotion to a target skin area, comprising:
one or more drivers, each of which drives one or more massage elements which providing massage vibrations in one or more ultrasonic and subsonic frequencies;
a signal generator that provides frequency references to said one or more drivers;
a power supply coupled to a contactless charger; and
a controller that provides commands to said one or more drivers, said signal generator and said power supply;
wherein said one or more drivers, said signal generator, said power supply and said controller are electrically and communicatively coupled together and are all physically enclosed in a device body.

2. The palm-held device of claim 1, further comprising an impedance match network coupled between each of said drivers and said one or more massage elements.

3. The palm-held device of claim 1, further comprising a feedback network coupled between said signal generator and said one or more massage elements.

4. The palm-held device of claim 1, further comprising:
a lotion release system, wherein said release system comprises one or more cartridges coupled to an array of dispensers embedded in surface of said one or more massage elements, wherein said array of dispensers are controlled by said controller.

5. The palm-held device of claim 1, wherein said signal generator provides audio frequencies, and wherein said one or more massage elements provide audio frequency vibrations.

6. The palm-held device of claim 1, wherein said signal generator comprises a crystal oscillator with a frequency that matches to said one or more massage elements' frequency.

7. The palm-held device of claim 1, wherein an output signal of said controller is used as an oscillating signal of said signal generator.

8. The palm-held device of claim 1, wherein said signal generator is a semiconductor circuit comprising a first inverter, a second inverter and a first capacitor that are electrically coupled in series, and an adjustable resistor electrically coupled to a first node between said first and second inverters and to a second node between said second inverter and said first capacitor.

9. The palm-held device of claim 1, wherein said signal generator is a semiconductor circuit comprising:
a non-inverting operational amplifier (NOA) with its input terminal coupled to a first feedback resistor via a third node, with its output terminal coupled to a fourth node and with its feedback terminal coupled to a second capacitor for AC coupling via a fifth node;
a second feedback resistor coupled between said fourth node and said third node, said third node being between said amplifier's input terminal and said first resistor; and
a third feedback resistor coupled between said fifth node and said amplifier's feedback terminal.

10. The palm-held device of claim 1, wherein each of said drivers is a semiconductor circuit comprising:
an input node;
a sixth node;
an output node;
an inverter coupled between said input node and said sixth node, said inverter comprising an n-type MOSFET and a p-type MOSFET with their gate terminals coupled to said input node and their source terminals coupled to said six node; and
an amplifier coupled between said sixth node and said output node; and
an inductor coupled between said output node and a power supply.

11. The palm-held device of claim 10, wherein said amplifier is any of:
an n-type MOSFET; and
a bipolar transistor.

12. The palm-held device of claim 1, wherein said one or more massage elements are driven at different frequencies alternatively in a time sequence determined by said controller.

13. An ultrasonic vibration generation device comprising:
one or more drivers, each of which drives one or more ultrasonic transducers which generate ultrasonic vibrations in one or more frequencies between 10 kHz to 100 MHz;
one or more signal generators that provide frequency reference signals to said one or more drivers;
at least one power supply coupled to said one or more drivers;
wherein said frequency reference signals being electrically decoupled from said one or more ultrasonic transducers; and
wherein said frequency reference signals being amplified by said one or more drivers and applied to said one or more ultrasonic transducers.

14. The palm-held device of claim 13, further comprising an impedance match network coupled between each of said drivers and said one or more massage elements.

15. The palm-held device of claim 13, further comprising a feedback network coupled between said signal generator and said one or more massage elements.

16. The palm-held device of claim 13, further comprising:
a lotion release system, wherein said release system comprises one or more cartridges coupled to an array of dispensers embedded in surface of said one or more massage elements, wherein said array of dispensers are controlled by said controller.

17. The palm-held device of claim 13, wherein said signal generator provides audio frequencies, and wherein said one or more massage elements provide audio frequency vibrations.

18. The palm-held device of claim 13, wherein said signal generator comprises a crystal oscillator with a frequency that matches to said one or more massage elements' frequency.

19. The palm-held device of claim 13, wherein an output signal of said controller is used as an oscillating signal of said signal generator.

20. A device for skin treatment by applying ultrasonic vibrations to a target skin area, comprising:
a plurality of ultrasonic transducers coupled to a skin treatment component with at least one surface of said skin treatment component being in contact with said target skin during a treatment session;
one or more drivers, each of which drives one or more ultrasonic transducers which generate ultrasonic vibrations in one or more frequencies between 10 kHz to 100 MHz;
one or more signal generators that provide frequency reference signals to said one or more drivers;
at least one power supply coupled to said one or more drivers; and
at least one controller that provides commands to said one or more drivers, said signal generator and said power supply;
wherein said frequency reference signals are amplified by said one or more drivers and applied to said one or more ultrasonic transducers;
wherein said frequency reference signals are electrically decoupled from said one or more ultrasonic transducers; and
wherein said one or more drivers, said signal generator, said power supply and said controller are electrically and communicatively coupled together and are all physically enclosed in a device body.
